(19) United States
(12) Patent Application Publication
(10) Pub. No.: US 2016/0106633 A1
(43) Pub. Date: Apr. 21, 2016

(54) SKIN CARE SHEET AND SKIN CARE ARTICLE

(71) Applicant: 3M INNOVATIVE PROPERTIES COMPANY, St. Paul, MN (US)

(72) Inventors: Hiroyasu Nagata, Kanagawa (JP); Eiichi Fujieda, Sagamihara (JP); Yukari Sekiya, Sagamihara (JP)

(21) Appl. No.: 14/894,753

(22) PCT Filed: May 30, 2014
(86) PCT No.: PCT/US2014/040146

§ 371 Date: Nov. 30, 2015

(30) Foreign Application Priority Data
May 30, 2013 (JP) 2013-114625

(51) Int. Cl. A61K 8/02; A61K 8/81; A61Q 19/10

(52) U.S. Cl. CPC A61K 8/0208; A61Q 19/10; A61K 8/8111

(57) ABSTRACT

A skin care sheet comprising a nonwoven fabric; the nonwoven fabric having a break elongation of at least 250%; the nonwoven fabric comprising an embossed section and a non-embossed section defined by the embossed section, the non-embossed pattern having a pitch of 1.2 to 10.0 mm; a proportion of a total area of the embossed section occupying from 5 to 30% of a total area of the nonwoven fabric on at least one surface of the nonwoven fabric; fibers present in the nonwoven fabric having a number-average fiber diameter of 0.1 to 15.0 μm; and the fibers containing a resin having a flexural modulus of 0.5 GPa or less, as well as a skin care article comprising the sheet, are provided.

### FIELD OF THE INVENTION

The present invention relates to a skin care sheet, and to a skin care article comprising the skin care sheet.

### BACKGROUND ART

Various types of nonwoven fabrics have conventionally been used in sheet-shaped skin care articles such as cleansing sheets. Nonwoven fabrics having a fiber diameter of, for example, about 20 to 40 μm have been widely used, but there is a demand for further improvement in skin care performance. In order to meet this demand, various special configurations, such as nonwoven fabrics (pulp, cotton, etc.) containing highly hydrophilic substances, have been proposed. Highly hydrophilic nonwoven fabrics are capable of retaining large amounts of aqueous liquid cosmetic components (such as aqueous cleansing agents), allowing diverse combinations of liquid cosmetic components and ordinary fibers to function as sheet-shaped skin care articles (such as cleansing sheets). Nonwoven fabrics with reduced fiber diameters have also been proposed. Nonwoven fabrics of fine fibers generally tend to exhibit good skin care performance (such as wiping performance).

To provide a skin care sheet having superior softness and texture and high liquid absorptivity, Japanese Unexamined Patent Application Publication No. 2006-288604A discloses a skin care sheet constituted by a nonwoven fabric capable of being impregnated with a liquid compound, the nonwoven fabric having an average fineness of 0.5 dtex or less and being formed from extremely slender fibers constituted by a water-insoluble thermoplastic resin, and containing a proportion of water soluble thermoplastic resin of 0.001 to 10 wt. % in the nonwoven fabric.

To provide a sheet for application to the skin that is comfortable to apply to the skin and exhibits good stability once applied, Japanese Unexamined Patent Application Publication No. 2009-228147A discloses a sheet for application to the skin that is a moist sheet formed by impregnating a layered sheet containing a hydrophilic fiber layer and an ultrafine fiber layer with from 100 to 1,500 parts by mass of a drug solution or a cosmetic per 100 parts by mass of the layered sheet, wherein the 10% elongation modulus strength in at least one direction of the moist sheet is in a range from 0.1 to 4.0 N/25 mm, and the elongation recovery in the same direction at 30% elongation is in a range from 30 to 100%.

To provide an elastic layered sheet that is comfortable when applied to an object and exhibits good stability once applied, and allows for the effects of a liquid component upon an object such as skin to be efficiently improved, Japanese Unexamined Patent Application Publication No. 2009-256856A discloses an elastic layered sheet layered by partially thermocompressing a layer of hydrophilic short fibers that are elastic in at least one direction and a layer of ultrafine fibers comprising (in terms of mass) at least 50 mass % long elastomer fibers of 15 μm diameter or less, wherein a discontinuous, regular ridge-and-valley shape caused by the partial thermocompression is formed on the surface of the hydrophilic short fiber layer exposed on one side of the sheet, the total area occupied by valleys on the surface of the hydrophilic short fiber layer is in a range from 3 to 40%, and the softening of at least long elastomer fibers in the layer of ultrafine fibers causes the fibers to bond to the fibers constituting the hydrophilic short fiber layer in the through-thickness direction of the sheet at the locations where the valleys are present, thereby integrating the hydrophilic short fiber layer and the ultrafine fiber layer.

### SUMMARY OF THE INVENTION

A nonwoven fabric constituted by ultrafine fibers (such as fibers having a number-average fiber diameter of 15 μm or less) tends to be capable of retaining large amounts of cosmetic components, and nonwoven fabrics constituted by such ultrafine fibers are capable of manifesting good skin care performance. However, the inventors discovered that conventional nonwoven fabrics constituted by ultrafine fibers impart an unpleasant slimy feeling during use when used as a skin care sheet, which is a factor deteriorating the feeling of use especially when used for wiping, as in the case of a cleansing sheet, and the insufficient softness of the fabric creates a scratching sensation upon the skin.

The present invention was conceived in order to solve the problems described above, and has an object of providing a skin care sheet that has a pleasant feel of use and skin feel (especially having reduced slimy and scratchy feelings) while exhibiting good skin care performance (such as wiping performance), as well as a skin care article comprising the sheet. The skin care sheet and skin care article provided by the present invention are advantageously applied, for example, to a cleansing sheet, a deodorant sheet, a facial mask, a lotion-impregnated sheet, a foundation-impregnated sheet, a sunscreen-impregnated sheet, or the like.

One aspect of the present invention provides: a skin care sheet comprising a nonwoven fabric; the nonwoven fabric having a break elongation of at least 250%; the nonwoven fabric comprising an embossed section and a non-embossed section defined by the embossed section, the non-embossed section having a pitch of 1.2 to 10.0 mm; a proportion of a total area of the embossed section occupying from 5 to 30% of a total area of the nonwoven fabric on at least one surface of the nonwoven fabric; the fibers present in the nonwoven fabric having a number-average fiber diameter of 0.1 to 15.0 μm; and the fibers containing a resin having a flexural modulus of 0.5 GPa or less.

Another aspect of the present invention provides a skin care article comprising the skin care sheet and a liquid cosmetic component.

In accordance with the present invention, a skin care sheet that has a pleasant feel of use and skin feel (especially having reduced slimy and scratchy feel) while exhibiting good skin care performance (such as wiping performance) and a skin care article comprising the sheet are provided.

### BRIEF DESCRIPTION OF THE DRAWINGS

FIGS. 1A to 1F are illustrations of examples of embossing (i.e., shapes of the embossed sections and non-embossed sections) provided on the skin care sheet according to the present invention.

FIG. 2 is an illustration of embossing (i.e., shapes of the embossed sections and non-embossed sections) provided in Comparative Example 14.

FIGS. 3A and 3B are microscopic images of the embossed shapes provided in Example 7 (FIG. 3A), and Example 11 (FIG. 3B).

FIG. 4 is an illustration of a method of measuring break elongation.

### DETAILED DESCRIPTION

A typical aspect of the present invention will now be described in detail, but the present invention is not limited thereto.

One aspect of the present invention provides a skin care sheet comprising a nonwoven fabric. The nonwoven fabric has a break elongation of about 250% or higher. The nonwoven fabric comprises an embossed section and a non-embossed section defined by the embossed section. That is, the nonwoven fabric is embossed. The non-embossed section has a pitch of about 1.2 mm to about 10.0 mm. The proportion of the total area of the embossed section occupies from about 5% to about 30% of the total area of the nonwoven fabric on at least one surface of the nonwoven fabric. The fibers present in the nonwoven fabric have a number-average fiber diameter of about 0.1 μm to about 15.0 μm. The fibers contain a resin having a flexural modulus of about 0.5 GPa or less.

Although not wishing to be bound by theories, the inventors hypothesized that resin grains formed by resin material hardening in lumps during the process of manufacturing the nonwoven fabric (hereafter also referred to simply as resin grains) and fused sections formed by fibers fusing to each other during the process of manufacturing the nonwoven fabric (hereafter also referred to simply as fused sections) are factors contributing to the unpleasant scratchy feel upon the skin during use exhibited by skin care sheets of conventional nonwoven fabrics, leading to poor skin feel. The resin grains and fused sections are believed to give rise to uneven nonwoven fabric density and thickness in the transverse direction (TD) of the nonwoven fabric sheet, and to breakage caused by stress concentration, thereby reducing the break elongation of the nonwoven fabric. Accordingly, reducing the incidence of resin grains and fused sections is believed to contribute to increased break elongation. The unraveling and stretching of the fibers when the nonwoven fabric is stretched that occurs when there is a paucity of resin grains and fused sections is also believed to contribute to increased break elongation. The inventors discovered that a nonwoven fabric in which the incidence of resin grains and fused sections has been reduced sufficiently to obtain a pleasant feel of use (in particular, reduced slimy and scratchy feelings) can be identified by the break elongation of the nonwoven fabric.

Reducing the incidence of resin grains and fused sections is also believed to contribute to satisfactory retention of various types of liquid cosmetic components, as described hereafter, and to satisfactory skin care performance (such as wiping performance).

From considerations of obtaining a satisfactory feel of use, the nonwoven fabric has a break elongation of at least about 250%. In a preferred aspect, the break elongation is at least about 270%, or at least about 300%. Meanwhile, from considerations of the workability of the nonwoven fabric, the break elongation is no more than about 600% or no more than about 500% in a preferred aspect. In a skin care sheet provided in one aspect of the present invention, a specific type of embossing is performed upon the nonwoven fabric, yielding superior effects due to the break elongation in the embossed state being in the range set forth above. In the present disclosure, “break elongation” is a value measured according to JIS L 1913 using a test piece having a width of 25 mm at a grip spacing of 25 mm and a tension rate of 300 mm/minute. More specifically, measurement can be performed according to the procedure set forth in the examples of the present disclosure, or according to a method understood as being comparable thereto by a person skilled in the art.

The nonwoven fabric can be single-layered or multi-layered. If multiple layers are present, the various properties of the nonwoven fabric may be exhibited by all of the individual layers, or by the multiple layers taken as a whole (especially in cases where the multiple layers are essentially inseparable barring damage to the nonwoven fabric). For instance, it is acceptable for either the break elongation of the individual layers or the break elongation of the multiple layers as a whole to fall within the prescribed range.

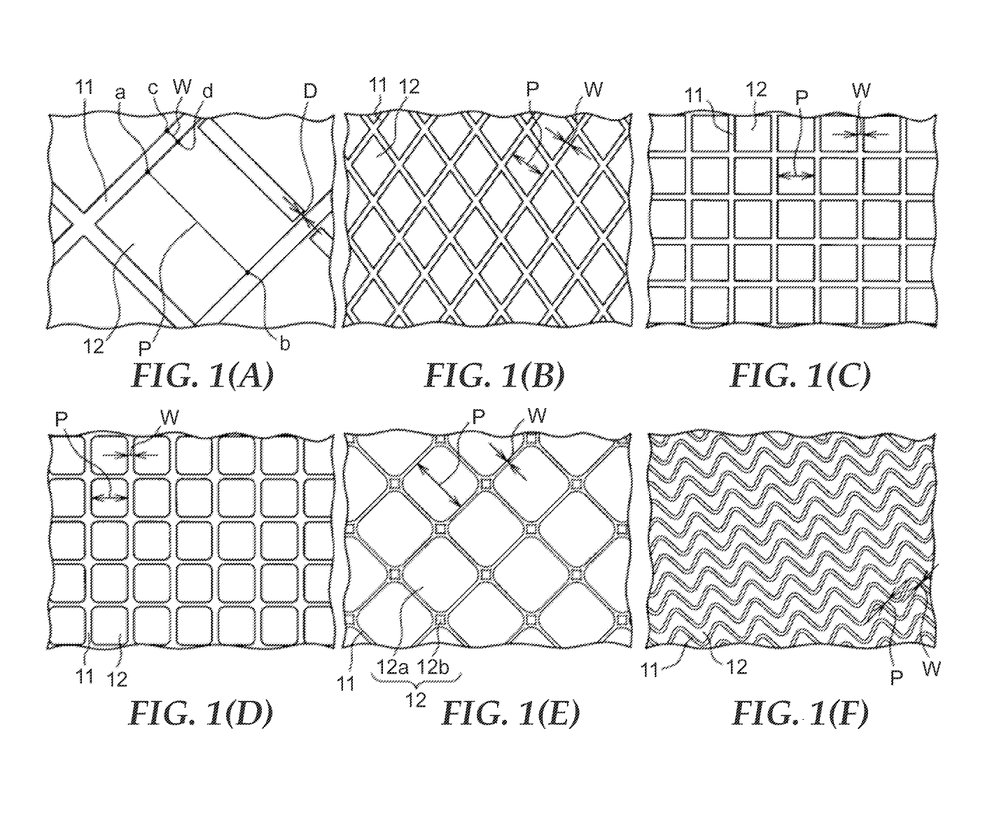

Exemplary aspects of the present invention will now be described with reference to the attached drawings, but the present invention is not limited to the following aspects. Identically labeled elements in the drawings operate in similar fashions. The dimensions shown in FIGS. 1A to 1F, 2, and 4 are not necessarily true dimension or strictly magnified versions of true dimensions.

FIG. 1 is an illustration of examples of embossing provided on the skin care sheet according to the present invention. The nonwoven fabric comprises an embossed section 11 and a non-embossed section 12 defined by the embossed section 11. Embossed patterns of various shapes are possible. Examples include the diamond shapes shown in FIGS. 1A and 1B, the square shape shown in FIG. 1C, or the roughly square shape in which the corners of a square have been rounded off shown in FIG. 1D. Further examples include the combination of different kinds of shapes shown in FIG. 1E, or the wavy pattern shown in FIG. 1F.

In the present disclosure, “embossed section” encompasses both (1) an area upon which embossing has been directly performed and (2) an area upon which embossing has not been directly performed, but that is an area of the nonwoven fabric that is surrounded by an embossed section and in which the thickness of the nonwoven fabric is no more than 25% of the maximum thickness of the nonwoven fabric (this kind of area being referred to as a “compressed section” in the present disclosure). In other words, the non-embossed section of the present disclosure is an area having a thickness greater than 25% and less than or equal to 100% of the maximum thickness of the nonwoven fabric. The compressed sections generally have a pitch of less than 1.2 mm (“pitch” defined identically as for the pitch of the non-embossed sections as described hereafter). For example, if the area 12b in FIG. 1E has a pitch of less than 1.2 mm, the area 12b is generally a compressed section, and the compressed section is not included in the non-embossed section.

The shape of the embossed section may be continuous or discontinuous on the surface of the nonwoven fabric. In a preferred aspect, the embossed section 11 separates a plurality of non-embossed sections 12, as shown, for example, in FIGS. 1A to 1E. That is, in the aspects shown in FIGS. 1A to 1E, the surface of the nonwoven fabric has an islands-in-the-sea shape in which the embossed section is the sea and the non-embossed sections constitute the islands. In the present disclosure, “sea” indicates that the section may be completely or substantially continuous, and “island” indicates an area surrounded by the “sea”. In the present disclosure, the shape of the embossed section being substantially continuous indicates that a discontinuous section is present between adjacent embossed sections, but the minimum distance between embossed sections facing each other over the discontinuous section is less than 1.2 mm. If a non-embossed section is surrounded by such a completely or substantially continuous embossed section, the non-embossed section is referred to as an “island”. If the embossed section is the sea and the non-embossed sections are islands, the plurality of non-embossed sections are completely or substantially separated from each other by the embossed sections.

For example, in a preferred aspect, the non-embossed sections are completely separated by a completely continuous embossed section (for example, see FIGS. 1B to 1E). In another preferred aspect, the non-embossed sections are substantially separated from each other by an embossed section that has discontinuous sections but is substantially continuous (for example, see FIG. 1A). In the latter case, the embossed section comprising discontinuous sections is disposed so that the minimum distance between embossed sections facing each other over the discontinuous sections (see, for example, distance D in FIG. 1A) is less than 1.2 mm so as to substantially separate the non-embossed sections. In another aspect, it is also possible for the embossed sections not to separate the non-embossed sections into islands. For example, as shown in FIG. 1F, the embossed sections and the non-embossed sections both have a wavy shape. In yet another example, a shape is possible in which the embossed sections are surrounded by a continuous non-embossed section, or by a non-embossed section in which a discontinuous section is present between adjacent non-embossed sections, but the minimum distance between non-embossed sections facing each other over the discontinuous section is less than 1.2 mm.

In the present disclosure, the non-embossed section has dimensions within a specific range described hereafter. In another preferred aspect, the embossed section has dimensions within a specific range described hereafter. Such specific manners of embossing contribute to the advantage of a pleasant feel of use (especially in terms of reduced slimy or scratchy feelings). This advantage can be obtained regardless of whether the nonwoven fabric is single-layered or multi-layered. However, the specific manners of embossing in the present invention particularly yield the advantage of a pleasant feel of use in skin care sheets in which the nonwoven fabric is single-layered.

If the embossed section constitutes the sea that completely or substantially separates a plurality of non-embossed sections from each other as islands, the nap and fuzziness of the nonwoven fabric will be reduced.

An aspect in which the embossed section extends in a direction that is diagonal with respect to the machine direction of the nonwoven fabric (i.e., that is neither horizontal nor vertical) is advantageous in terms of wiping performance, as the nonwoven fabric is generally moved up and down in either the machine direction or the transverse direction of the nonwoven fabric during actual wiping. Examples of such embossed sections are shown in FIGS. 1A, 1B, 1E, and 1F, viewing the left-to-right direction in the drawings as the machine direction.

The non-embossed section has a pitch of about 1.2 mm to about 10.0 mm. In the present disclosure, the pitch of the non-embossed section is measured as follows.

(1) If non-embossed sections form the islands:

In FIG. 1A, the length of a line segment drawn from a point a on an edge of the non-embossed section to another point b on another edge of the non-embossed section on a line that is normal to the first edge and passes only through the interior of the non-embossed section is taken as the pitch P. The line segment drawn from point a overlaps point b, and a line segment similarly drawn from point b along a normal line overlaps point a.

The pitch is obtained by dividing the skin care sheet into four sections of equal area, selecting and measuring five pairs of points a and b in non-embossed sections in the divided four sections, and finding the numerical average of the measured values. That is, the pitch value intended in the present disclosure is the pitch value found by obtaining the numerical average of the pitches measured for five pairs times four areas in the prescribed range set forth above, for a total of 20 pairs.

The pitch of non-embossed sections of shapes such that two points a, b cannot be selected (such as triangles or star shapes) is defined as the maximum distance across the non-embossed section. In the present disclosure, “distance across” refers to the maximum length of a line segment drawn from a point a1 on an edge of one non-embossed section passing over a different point b1 on another edge of the non-embossed section without passing outside the non-embossed section. In the present disclosure, “maximum distance across” refers to the maximum value out of multiple distances across measured from different points a1 in one non-embossed section. Accordingly, the pitch in this case is the value measured for a single location on a single non-embossed section. The maximum distance across is measured over the entire skin care sheet, and the numerical average of the measured values is taken as the pitch.

If, for example, the outer edge of the non-embossed section has the shape of an equilateral triangle, the pitch is the length of a perpendicular line drawn from one selected apex to the side opposite the apex. If the outer edge of the non-embossed section has the shape of a star shape, the pitch is, for example, the distance between P1 and P3 when the five points of the star shape are labeled P1, P2, P3, P4, and P5.

(2) If non-embossed sections do not form the islands (e.g., FIG. 1F):

A first embossed section is arbitrarily selected as a reference embossed section. Next, the embossed section out of the embossed sections adjacent to the first embossed section that has the minimum distance from the first embossed section is selected as a second embossed section. If the minimum distance D1 between the first embossed section and the second embossed section is less than 1.2 mm, the two embossed sections are considered to be substantially continuous. Thus, if D1 is 1.2 mm or greater, the value for D1 is adopted as the pitch of the non-embossed section. Next, using the second embossed section as a reference embossed section, the embossed section (except for the first embossed section) out of the embossed sections adjacent to the second embossed section that has the minimum distance from the second embossed section is selected as a third embossed section. If the minimum distance D2 between the second embossed section and the third embossed section is 1.2 mm or greater, the value for D2 is also adopted as the pitch of the non-embossed section. Measurement is performed in a similar manner until no embossed sections remain to be selected. The numerical average of the values obtained for D1 to Dn (where n is the number of values measured) is defined as the pitch of the non-embossed section.

If pitch cannot be determined using only one of the methods described above due to the non-embossed section comprising a variety of shapes, the pitch of the various parts of the non-embossed section is measured using the appropriate method for each, after which the results are multiplied by the proportion of the area of each part to calculate an average value.

An example in which (i) a shape in which two points a, b can be selected and (ii) a shape in which two points a, b cannot be selected are both present in (1) above will be described. The pitch of the non-embossed section is calculated according to the formula: pitch=(PA×A+PB×B)/100; wherein A is the percentage out of 100 mass % of the total area of the non-embossed section occupied by part (i), B is the percentage occupied by part (ii), PA is the pitch calculated for part (i) alone according to the method described above, and PB is the pitch calculated for part (ii) alone according to the method described above.

In order to avoid fiber fusion in the parts of the non-embossed section near the embossed section and obtain a soft feel against the skin, and to obtain, for example, satisfactory wiping performance when the sheet is used as a cleansing sheet, the pitch of the non-embossed section is about 1.2 mm or higher, or, preferably, about 1.8 mm or higher or about 2.3 mm or higher. In order to prevent a film from forming in the non-embossed section (i.e., when the gaps between fibers break down and the unevenly textured surface of the nonwoven fabric becomes flat), to reduce slimy or scratchy feelings, and to obtain satisfactory wiping performance when, for example, the sheet is used as a cleansing sheet, the pitch of the non-embossed section is about 10.0 mm or less, preferably about 8.5 mm or less, or about 7.5 mm or less.

The non-embossed section may comprise a plurality of shapes, as in non-embossed sections 12a, 12b shown in FIG. 1E. If the pitch of the non-embossed section 12b falls outside the range prescribed for the present invention, that area of the nonwoven fabric will generally have a thickness that is no more than 25% of the maximum thickness of the nonwoven fabric; thus, this section is defined as an embossed section rather than as a non-embossed section. Such an area having a thickness of 25% or less of the maximum thickness of the nonwoven fabric even though embossing has not been directly performed thereupon may acceptably constitute, for example, about 10% or less, or about 7% or less, of the total area upon which embossing has not been directly performed.

In a preferred aspect, the embossed section is an embossed line. In the present disclosure, “embossed line” generally refers to all forms that will be understood by a person skilled in the art as constituting a line due to the embossed section extending continuously or discontinuously in an elongated shape. If the embossed section extends discontinuously, it is defined as an embossed line if the space between adjacent embossed sections is 1.2 mm or less. The embossed line may be a straight line or a curved line, and the width thereof may be uniform or vary in different parts. In a preferred aspect, the embossed section is an embossed line as shown, for example, in FIGS. 1A to 1F. In a preferred aspect, the width W of the embossed line is about 0.1 mm to about 1.3 mm. The width W is measured as follows. Referring, for example, to FIG. 1A, the length of a line segment extending from a point c on an edge of the embossed line along a line normal to the edge through only the interior of the embossed line to another point d on another edge of the embossed line is taken as the width W. The line segment drawn from point c overlaps point d, and a line segment drawn from point d overlaps point c. For each embossed section, the value for width is found by selecting and measuring ten pairs of points c and d for the embossed section (the points being selected at uniform intervals along the lengthwise direction of the embossed line, and areas in which multiple embossed lines overlap being excluded), and finding the numerical average of the measured values. If the embossed line is constituted by discontinuous embossed sections, 10 pairs of points c and d are selected for each embossed section as described above (the points being selected at uniform intervals along the lengthwise direction of the embossed line, and areas in which multiple embossed lines overlap being excluded) to measure the width.

In a preferred aspect, the width of the embossed line is about 0.1 mm or greater, preferably about 0.2 mm or greater or about 0.4 mm or greater, in order to satisfactorily reduce slimy feeling. In a preferred aspect, in order to prevent a film from forming in the non-embossed section (i.e., when the gaps between fibers break down and the unevenly textured surface of the nonwoven fabric becomes flat), to reduce slimy or scratchy feelings, and to obtain satisfactory wiping performance when, for example, the sheet is used as a cleansing sheet, the width of the embossed line is about 1.3 mm or less, preferably about 1.0 mm or less, or about 0.8 mm or less.

The proportion of the total area of the embossed section relative to the total area of the nonwoven fabric (referred to as the “embossed area proportion” in the present disclosure; also includes those areas that are counted as part of the embossed section due to having a thickness of 25% or less of the maximum thickness of the nonwoven fabric, despite embossing not having been applied directly thereto) is from about 5% to about 30% on at least one surface of the nonwoven fabric. In order to obtain a pleasant feel of use, the embossed area proportion is about 5% or greater, preferably about 10% or greater, or about 17% or greater; in order to prevent the nonwoven fabric from becoming excessively hard, the embossed area proportion is about 30% or less, preferably about 23% or less, or about 18% or less.

In the present disclosure, the pitch of the non-embossed section, the width of the embossed line, and the embossed area proportion are each analyzed in the depth direction using an optical microscope according to the procedure set forth below. As discussed above, parts having a thickness of 25% or less of the thickness of the part of maximum thickness are defined as embossed sections, and an image measuring procedure commonly used by persons skilled in the art is employed. Specifically, in the present disclosure, locations having a thickness that is less than or equal to the thickness prescribed above, even if embossing has not been applied directly thereto, are considered part of the embossed section along with locations upon which embossing has been directly applied.

More specifically, an optical microscope (such as a Keyence VHX-1000) is used to observe a nonwoven fabric sample that had been dried at 40° C. in a dry oven for 72 hours at a magnification of 100× or 50× according to the measurement menus with which the optical microscope is equipped. Upper and lower limits are set so as to include the most prominent part of the surface of the nonwoven fabric (i.e., the position of maximum thickness) and the most recessed part of the surface (i.e., the position of minimum thickness), and the thickness in the depth direction of the nonwoven fabric sample is visualized. Parts having 25% or less the thickness relative to the thickness of the most prominent part of the surface are counted as embossed sections, and the pitch of the non-embossed section, and the width of the embossed section if it is an embossed line, are measured using the scale with which the microscope is equipped. The embossed area proportion is measured using the area measurement tool included in the measurement menus. More specifically, measurements can be performed according to the procedure set forth in the examples of the present disclosure, or according to a method understood as being comparable thereto by a person skilled in the art.

The fibers present in the nonwoven fabric have a number-average fiber diameter of about 0.1 μm to about 15.0 μm. In order to reduce slimy textures, the number-average fiber diameter is about 0.1 μm or greater, preferably about 0.5 μm or greater, or about 1.0 μm or greater. For the sake of softness of texture and satisfactory skin care performance (such as wiping performance of, for example, a cleansing sheet), the number-average fiber diameter is about 15.0 μm or less, preferably about 12.0 μm or less, or about 10.0 μm or less. In the present disclosure, the number-average fiber diameter is the numerical average value measured at ten locations via image analysis in an image observed at 350× using a scanning electron microscope. The fibers may have a circular cross-sectional shape or a so-called modified cross-sectional shape (such as triangular, rectangular, hexagonal, star shape, ellipsoid, or similar cross section), with the fiber diameter being measured at the point of maximum diameter. In a preferred aspect, the fibers have a roughly circular cross-sectional shape for the sake of skin feel.

In a preferred aspect, the nonwoven fabric is a long-fibered nonwoven fabric. A long-fibered nonwoven fabric is advantageous in terms of skin feel. The long-fibered nonwoven fabric can be manufactured according to a procedure known to a person skilled in the art using a spun bonding method, especially a melt-blowing method.

The fibers can be constituted by one or two or more materials. If two or more materials are used, examples of fibers include sheath-core fibers, mixed fibers, and fibers obtained by spinning a melted mixture of two or more components.

The fibers contain a resin having a flexural modulus of about 0.5 GPa or less. A resin of such a low modulus is advantageous in terms of softness of texture against the skin. In a preferred aspect, the flexural modulus of the resin is about 0.4 GPa or less, about 0.3 GPa or less, or about 0.1 GPa or less. Also, in a preferred aspect, the flexural modulus of the resin is about 0.001 GPa or greater, about 0.01 GPa or greater, or about 0.02 GPa or greater in order to obtain satisfactory durability for the nonwoven fabric. In a preferred aspect, the proportion of the resin having a flexural modulus in the abovementioned prescribed range out of the total mass of resin in the fibers is about 20 mass % or higher, about 50 mass % or higher, or about 100 mass %. In another preferred aspect, the arithmetic average of the flexural modulus of all the resin in the fiber is within the abovementioned prescribed range. In the present disclosure, the flexural modulus is as measured according to ASTM D790.

In a preferred aspect, the resin contains a thermoplastic resin. A thermoplastic resin is advantageous in allowing a nonwoven fabric having the anticipated properties of the present invention to be easily manufactured. Examples of thermoplastic resins include olefin-based, urethane-based, ester-based, amide-based, styrene-based resins, and the like.

Examples of olefin-based resins include polyethylene, ethylene/alpha-olefin copolymer, ethylene/glycidyl methacrylate copolymer, ethylene/methyl methacrylate copolymer, ethylene/styrene copolymer, polypropylene, maleic acid-modified polypropylene, propylene/styrene copolymer, polybutylene, and the like. Polyolefin may be a crystalline or an amorphous polyolefin.

Examples of polyethylenes include low density polyethylene (LDPE), linear low density polyethylene (LLDPE), high density polyethylene (HDPE), and the like. Examples of polypropylenes include propylene homopolymers, propylene-based bipolymers, and propylene-based tripolymers. In a preferred aspect, the polyolefin is crystalline polypropylene, low-crystalline (e.g., crystallinity: about 45 to 55%), or linear low density polyethylene (LLDPE).

Any crystalline polypropylene is acceptable without particular restriction as long as it exhibits hard elasticity. Preferable examples of crystalline polypropylenes include propylene homopolymers, propylene-based ethylene copolymers, propylene-based alpha-olefin copolymers, and the like.

Linear low density polyethylene (LLDPE) is a copolymer of ethylene, the main component, and slight amounts of alpha-olefin, typically produced using a coordinated anionic polymerization catalyst such as a Ziegler-Natta catalyst. Linear low density polyethylene generally has a density of about 0.910 to 0.925 (per JIS K 7112); linear low density polyethylene with low density, low-crystallinity will exhibit little post-elongation warping. Accordingly, the density is preferably 0.915 g/cm³ or higher, and 0.940 g/cm³ or less. Examples of alpha-olefins which are copolymerization monomers include alpha-olefins having from 4 to 8 carbons, such as 1-butene, 1-hexene, 4-methylpentene, and 1-octene.

In a preferred aspect, the polyolefin is a polyolefin-based elastomer. An ethylene/alpha-olefin copolymer produced using a metallocene as a catalyst is preferable as the polyolefin-based elastomer for the sake of workability, costs, lightfastness, chemical resistance, and skin sensitivity. Examples of the alpha-olefin copolymerized with ethylene in an ethylene/alpha-olefin copolymer include alpha-olefins having from 3 to 30 carbons, such as propylene, 1-butene, 1-pentene, 1-hexene, 1-octene, 1-heptene, 4-methyl-1-pentene, 4-methyl-1-hexene, 4,4-dimethyl-1-pentene, and octadecene. Of these, 1-hexene, 1-octene, 1-heptene, 4-methyl-1-pentene are preferably used. The blended proportions of ethylene and alpha-olefin in the ethylene/alpha-olefin copolymer are preferably about 40 mass % or greater and about 98 mass % or less for ethylene, and about 2 mass % or greater and about 60 mass % or less for the alpha-olefin. Examples of preferred polyolefin-based elastomers include syndiotactic poly(1,2-butadiene), poly(trans-1,4-isoprene), and the like.

Examples of urethane-based resins include nylon 6, nylon 66, and the like. In a preferred aspect, the polyurethane is a polyurethane-based elastomer. Polyurethane-based elastomers are polymers containing a urethane bond in a molecule, and are generally obtained through a polyaddition reaction between a polyol such as a long chain polyol or a short chain polyol, and an isocyanate such as diisocyanate. In a polyurethane-based elastomer, the long chain polyol is a flexible component, and the short chain polyol and diisocyanate are molecule-binding components. Typical polyols used as raw materials in polyurethane-based elastomers include polyester-based polyols (adipate-based, polycaprolactone-based, etc.) and polyether-based polyols.

Examples of long chain polyols include polyether diols (such as poly(oxytetramethylene)glycol and poly(oxypropylene)glycol), and polyester diols (such as poly(ethylene adipate)glycol, poly(1,4-butylene adipate)glycol, poly(1,6-hexylene adipate)glycol, poly(hexanediol-1,6-carbonate)glycol), and the like.

Examples of short chain polyols include ethylene glycol, 1,3-propylene glycol, bisphenol A, 1,4-butanediol, 1,4-hexanediol, and the like.

Examples of diisocyanates include 4,4′-diphenylmethane diisocyanate, toluene diisocyanate, hexamethylene diisocyanate, and the like, and any of an aromatic isocyanate, an aliphatic isocyanate, or a cycloaliphatic isocyanate can be used.

Examples of ester-based resins include polyethylene terephthalate, polybutylene terephthalate, and polyester-based elastomers. Examples of polyester-based elastomers include those constituted by a block having an aromatic polyester as a hard segment and a block having an aliphatic polyether or an aliphatic polyester as a soft segment.

Examples of styrene-based resins include polystyrene-based elastomers. Examples of polystyrene-based elastomers include styrene-ethylene/butylene-styrene block (SEBS) copolymers, styrene-ethylene/propylene-styrene (SEPS) block copolymers, styrene-ethylene/butylene-ethylene (SEBC) block copolymers, ethylene-ethylene/butylene-ethylene (CEBC) block copolymers, and the like. If an SEBS copolymer is used as a polystyrene-based elastomer, the proportion of styrene per total 100 mass % of the SEBS copolymer is preferably at least 10 mass %, and preferably no more than 25 mass %.

In a preferred aspect, the thermoplastic resin is an olefin-based resin. In the present disclosure, the term “-based” as used in connection with types of resins indicates that the named repeating unit constitutes more than 50 mass % of the total mass of repeating units. For example, an “olefin-based resin” is a resin in which olefin repeating units constitute more than 50 mass % of the total repeating units.

In a preferred aspect, the thermoplastic resin includes an elastomer. An elastomer is advantageous in terms of softness of texture. Examples of elastomers include olefin-based, urethane-based, ester-based, amide-based, styrene-based, vinyl chloride-based, and similar elastomers, with more specific examples being those listed above.

In a preferred aspect, the thermoplastic resin is an olefin-based elastomer. An olefin-based elastomer is especially advantageous in terms of softness of texture and workability. A combination of an olefin-based elastomer and another olefin-based resin is preferable. In such cases, the olefin-based resin contributes to improved hardness and workability.

The resin may be a homopolymer, a copolymer, or a blend of two or more types of resins. Preferred examples of copolymers include ethylene/butylene copolymers, ethylene/propylene copolymers, ethylene/octene copolymers, and the like. A preferred example of a blend is an ethylene/octene copolymer or the like.

In a preferred aspect, the fibers contain about 25 mass % or greater, about 50 mass % or greater, or about 75 mass % or greater of a resin having a melting point of about 80° C. to about 130° C. In order to reduce fiber fusion caused by the resin temperature being too high with respect to the melting point of the resin and fluidity being too high when, for example, resin fibers are sent to a collector roller during the process of manufacturing the nonwoven fabric, and to reduce slimy textures, the melting point is, for example, about 80° C. or higher, about 85° C. or higher, or about 90° C. or higher, and, for the sake of ease of manufacture, about 130° C. or less, about 125° C. or less, or about 120° C. or less. The melting point is as measured via differential scanning calorimetry, and is measured from the peak top of the heat absorption peak obtained via differential thermal analysis.

In a preferred aspect, the fibers contain about 25 mass % or greater, about 50 mass % or greater, or about 75 mass % or greater of a resin having a melt flow rate at 190° C. of about 10 g/10 min or greater and about 1,500 g/10 min or less. In a preferred aspect, the melt flow rate at 190° C. is about 10 g/10 min or higher, about 15 g/10 min or higher, about 20 g/10 min or higher, or about 30 g/10 min or higher for the sake of ease of spinning. In order to prevent the fibers from becoming fragile and stably manufacturing the nonwoven fabric, the melt flow rate at 190° C. is no more than about 1,500 g/10 min or no more than about 300 g/10 min. The melt flow rate is as measured according to ASTM D1238.

The fiber may contain various additives generally used to manufacture nonwoven fabrics, as desired, such as antioxidants, UV absorbers, weatherproofing agents, anti-blocking agents, lubricants, colorants, inorganic fillers, oils, and the like. For example, a thermoplastic plastic, an oil, or the like can be used as an additive in order to improve the molten fluidity of the elastomer. Such additives can be introduced at any time during the process of manufacturing the nonwoven fabric. A surface treatment such as a hydrophilizing treatment may also be performed upon the nonwoven fabric as desired. A hydrophilizing treatment, for example, can be performed according to a conventionally known typical method, such as by immersing the nonwoven fabric in a hydrophilizing agent, plasma treating the nonwoven fabric, or the like.

In a preferred aspect, the thickness of the nonwoven fabric is about 0.3 mm or greater, or about 0.4 mm or greater, for the sake of liquid retention and ease of use, and about 2.0 mm or less, or about 1.5 mm or less, for the sake of ease of packaging. In the present disclosure, “thickness” refers to the value measured for a nonwoven fabric sample dried at 40° C. in an oven for 72 hours using an optical microscope (such as a Keyence VHX-1000) at a magnification of 100×, 50×, or 25×, using the scale included with the optical microscope, and is the maximum thickness of the nonwoven fabric excluding raised fibers and the like (i.e., the thickness of the most prominent part of the surface).

In a preferred aspect, the nonwoven fabric has a basis weight of about 30 gsm or greater, about 40 gsm or greater, or about 45 gsm or greater, for the sake of liquid retention, durability, and ease of use, and about 200 gsm or less, about 150 gsm or less, or about 100 gsm or less, for the sake of liquid retention, softness of texture, ease of use, and ease of packaging.

In a preferred aspect, there is no particular restriction upon the shape and size of the skin care sheet; a shape and size that are suitable or convenient for applying a cosmetic component to the skin can be used. For example, if the sheet is used as a cleansing sheet, the area of, for example, one primary surface can be about 400 cm² to about 100 cm², about 350 cm² to about 125 cm², or about 300 cm² to about 150 cm².

In one aspect, the skin care sheet can be constituted solely by the nonwoven fabric described above (hereafter also referred to as “the essential nonwoven fabric”). In another aspect, the skin care sheet can be constituted by a laminate of a combination of the essential nonwoven fabric and an additional layer. Each of the essential nonwoven fabric and the additional layer can comprise one or more layers. The essential nonwoven fabric and the additional layer can be disposed in any way in the laminate, but the essential nonwoven fabric should at least be disposed as the outermost surface layer of the laminate so that the essential nonwoven fabric contacts the skin. Examples of additional layers include an additional nonwoven fabric (lacking one or more of the essential properties of the essential nonwoven fabric described above), a backing film, a layer of water-absorbent resin, a layer of pulp, and the like. A more specific example of an additional layer is a rayon spunlace nonwoven fabric. In a preferred aspect, the thickness of the additional layer is about 0.1 mm or greater, about 0.2 mm or greater, or about 0.3 mm or greater, for the sake of satisfactorily obtaining the desired effects from the additional layer, and the thickness of the additional layer is about 1.0 mm or less, about 0.8 mm or less, or about 0.6 mm or less, for the sake of satisfactorily maintaining the effects of the essential nonwoven fabric.

In a preferred aspect, the skin care sheet is a single-layered nonwoven fabric constituted by a single layer of the essential nonwoven fabric. Such a skin care sheet is preferable, as the desired effects can be sufficiently obtained from a single layer, and such a sheet is advantageous in terms of ease of manufacturing and manufacturing costs. The actions of the specific embossing performed upon the skin care sheet yield a satisfactory feel of use even using a single layer.

The nonwoven fabric can be manufactured by various methods using the materials described above. Typical methods of manufacturing nonwoven fabrics include a dry method, a wet method, a spun-bond method (including melt-blowing and other methods), an air-laid method, and the like. In a preferred aspect of the present invention, a spun-bond method, especially melt-blowing, is used for the sake of easily manufacturing a nonwoven fabric having the desired fiber diameter. A process of manufacturing the nonwoven fabric will now be described using melt-blowing as an example.

A conventionally known melt-blowing process can be used. Ordinarily, in melt-blowing, heated molten raw resin is extruded from a die as molten fibers while hot air is blown thereupon at high speed to form ultrafine fibers, which are trapped on a collector (such as a net) to obtain a web. The web may be, optionally, compressed through calendering or the like to the desired basis weight.

In an exemplary aspect, a melt-blowing apparatus comprises a die having a set of positioned, aligned, parallel die orifices. The die orifices open from a central die cavity. The diameter of the orifices, the number of orifices per linear centimeter on the front surface of the die, the length of the orifices, and the like can be designed as appropriate according to purpose. The polymer is introduced into the central die cavity and die orifices from a melt extruder comprising a resin hopper, a barrel, and a screw within the barrel. Molten resin exits the barrel of the extruder and enters a gear melt pump, which improves control of the flow of molten polymer passing through a component element downstream of the apparatus. After leaving the pump, the molten resin flows into a die comprising the die cavity, through which is sent a liquid fiber-forming material. The fiber-forming polymer is extruded from the die orifices into a fiber-forming hot air current. The fiber-forming current is kept at high speed, and exits orifices or slots at both sides of the set of die orifices. The high-speed air current is provided to the slots from two peripheral cavities.

Fibers finely formed by the high-speed hot air from the slots after leaving the die orifices are collected from the die or on a trapping device such as a belt disposed a certain distance from the die. The distance can be set according to the crystallization behavior of the polymer, how swiftly the polymer is cooled to a completely non-tacky state, and other processing conditions. The trapping device can be a flat screen, drum, cylinder, or a microporous screen belt. The cylinder drives a belt. A degassing device is disposed behind the porous trapping device, and facilitates the trapping of the fibers as a web on the screen or other porous trapping device. The web travels from the trapping device to embossing rollers (either one of a pair of rollers may lack a pattern), where it is embossed. The embossing rollers can also be used off-line.

A more detailed melt-blowing procedure is disclosed in detail in, for example, Japanese Unexamined Patent Application Publication (Translation of PCT Application) No. 2004-528070A, to which a person skilled in the art is directed.

If the die temperature during melt-blowing is increased in order to reduce the fiber diameter, the fibers will fuse to each other on the collector. The die temperature must be reduced in order to prevent fusion, but this makes it difficult to achieve a fine fiber diameter. The inventors discovered that controlling the die temperature, hot air temperature, and/or hot air pressure is especially effective in order to minimize the formation of fused sections between resin grains and fibers in the nonwoven fabric to obtain the desired break elongation, and thus a pleasant feel of use, while achieving the desired small fiber diameter. Specifically, it is preferable to control a combination of one or two or more of the die temperature, the hot air temperature, and the hot air pressure in the following manners. It is more preferable to control the combination of the die temperature, the hot air temperature, and the hot air pressure in the following manners.

In a preferred aspect, the die temperature is about 180° C. or higher, about 200° C. or higher, or about 220° C. or higher, for the sake of avoiding an excessively large fiber diameter and obtaining a pleasant feel of use and satisfactory skin care performance, and about 400° C. or less, about 390° C. or less, or about 380° C. or less, for the sake of avoiding the formation of fused sections between resin grains and fibers, and obtaining a pleasant feel of use.

In a preferred aspect, the hot air temperature is about 380° C. or less, about 360° C. or less, or about 340° C. or less, for the sake of avoiding the formation of fused sections between resin grains and fibers, and obtaining a pleasant feel of use.

In a preferred aspect, the hot air pressure is about 1 kPa or higher, about 3 kPa or higher, or about 5 kPa or higher, for the sake of avoiding an excessively large fiber diameter and obtaining a pleasant feel of use and satisfactory skin care performance, and about 150 kPa or less, about 120 kPa or less, or about 100 kPa or less, for the sake of avoiding the formation of fused sections between resin grains and fibers, and obtaining a pleasant feel of use.

An embossed section and a non-embossed section of the desired shape is formed by embossing one sheet of web, or two or more overlapped webs, obtained according to the procedures described above. Embossing can be performed using any conventionally known method, such as by passing a web through a pair of embossing rollers and partially thermocompressing the web to form the desired embossed section. One of the pair of embossing rollers can be flat and the other can have a raised section corresponding to the shape of the embossed section. The temperature of the embossing roller (i.e., the embossing temperature) can be adjusted, for example, according to the melting point of the resin which is the material forming the nonwoven fabric. For example, in a preferred aspect, the temperature is ±40° C. with respect to the melting point of the resin. The embossing temperature can be, for example, about 40° C. or higher, about 50° C. or higher, or about 60° C. or higher, and, for example, about 170° C. or less, about 160° C. or less, or about 150° C. or less. The embossing pressure can be, for example, about 0.01 MPa or greater, about 0.05 MPa or greater, or about 0.1 MPa or greater, and, for example, about 20 MPa or less, about 15 MPa or less, or about 10 MPa or less.

The nonwoven fabric can be manufactured according to a process such as that described above. If the skin care sheet further comprises an additional layer, the essential nonwoven fabric and the additional layer can be integrated (for example, compression bonded) using any conventionally known method to manufacture a laminate. The skin care sheet can be manufactured as described above.

The skin care sheet can be used for various skin care article applications involving the application of a cosmetic component via contact with the skin, such as cleansing sheets, deodorant sheets, facial masks, lotion-impregnated sheets, foundation-impregnated sheets, and sunscreen-impregnated sheets. The advantages of the present invention are especially prominent in applications in which the sheet is rubbed against the skin (especially sensitive facial skin), such as cleansing sheets, deodorant sheets, lotion-impregnated sheets, foundation-impregnated sheets, and sunscreen-impregnated sheets. The skin care sheet provided by the present invention is especially advantageous in terms of wiping performance, and is useful as a cleansing sheet exhibiting good cleansing performance upon makeup such as foundation, lipstick, eyeshadow, or mascara.

The skin care sheet can be combined with a cosmetic component according to purpose. The skin care sheet can be combined with a cosmetic component by impregnating the skin care sheet with a cosmetic component for use, or an article such as the skin care article described hereafter can be provided as a pre-prepared combination of the skin care sheet and a cosmetic component. Various components can be used as the cosmetic component, such as the liquid cosmetic components described hereafter.

Another aspect of the present invention provides a skin care article comprising the skin care sheet according to the present invention described above and a liquid cosmetic component. In the present disclosure, “liquid cosmetic component” generally encompasses components that have a sufficient level of fluidity at usage temperature to allow for application to the skin; accordingly, the invention is not limited to embodiments comprising only liquids, but may also be an embodiment in which solids are dispersed within a liquid. The liquid cosmetic component may be a solution, emulsion, suspension, or the like.

In a typical skin care article, a liquid cosmetic component is retained in the skin care sheet via adsorption or absorption. That is, the skin care sheet is impregnated with a liquid cosmetic component.

The liquid cosmetic component may be aqueous or oleaginous. The component may also be an oil-in-water emulsion or a water-in-oil emulsion.

In an aqueous liquid cosmetic component, one or a combination of two or more hydrophilic media, such as water, a monohydric alcohol (such as ethanol or propanol), or a dihydric alcohol (such as ethylene glycol or propylene glycol), can be used. Water and/or ethanol are preferred hydrophilic media due to causing little irritation to the skin.

In an oleaginous liquid cosmetic component, one or a combination of two or more types of oleophilic media can be used, such as higher fatty acids (such as lauric acid, myristic acid, palmitic acid, stearic acid, or oleic acid), vegetable oils (such as jojoba oil, olive oil, palm oil, camellia oil, macadamia nut oil, avocado oil, corn oil, sesame oil, wheat germ oil, linseed oil, castor oil, grapeseed oil, safflower oil, sunflower oil, hazelnut oil, rosehip oil, argan oil, or botanical squalene), animal oils (such as lanolin, horse fat, or animal squalane), and mineral oils (such as liquid paraffin, microcrystalline wax, or silicone oil).

The liquid cosmetic component can contain one or more types of cosmetic active ingredients generally used in the skin care field according to purpose. Examples of active ingredients include surfactants, moisturizers, emollients, anti-chafing agents, skin softeners, astringents, skin-lightening agents, antioxidizing agents, anti-inflammatory agents, preservatives, UV blockers, stabilizers, viscosity adjusting agents, emulsifiers, fragrances, colorants, solubilizers, various powders, vitamins, and the like. For example, if the sheet is used as a cleansing sheet, a surfactant, moisturizing agent, or the like can be used as an active ingredient. Examples of surfactants include carboxylate salt-based, sulfonate salt-based, sulfuric acid ester-based, and other types of anionic surfactants, quaternary ammonium salts and other cationic surfactants, amino acid-based, betaine-based, and other types of amphoteric surfactants, and ester-based, ether-based, ester/ether-based, and other non-ionic surfactants.

The total amount of other components per 100 parts by mass medium in the liquid cosmetic component is, for example, about 0.1 parts by mass or greater, about 0.5 parts by mass or greater, or about 1 part by mass or greater, and, for example, about 80 parts by mass or less, about 75 parts by mass or less, or about 70 parts by mass or less.

The amount of liquid cosmetic component per 100 parts by mass of the skin care sheet in the skin care article is, for example, about 0.1 parts by mass or greater, about 0.5 parts by mass or greater, or about 1 part by mass or greater, and, for example, about 80 parts by mass or less, about 75 parts by mass or less, or about 70 parts by mass or less.

Any conventionally known method can be used to manufacture a skin care article using the skin care sheet, such as a method that immerses the skin care sheet in a liquid cosmetic component bath, or a method that fills the skin care sheet with a liquid cosmetic component.

The break elongation of the nonwoven fabric in the skin care article can be measured using a similar method as that used to measure the break elongation of the skin care sheet described above after the nonwoven fabric has been completely dried. In general, the nonwoven fabric used in the skin care sheet described above and a nonwoven fabric obtained by impregnating the nonwoven fabric with a liquid cosmetic component to create a skin care article, followed by completely drying the skin care article, will have comparable break elongations. In the present disclosure, “completely dried” indicates the state achieved by drying the skin care article in a dry oven at 40° C. for 72 hours.

### EXAMPLES

The present invention will now be described in further detail using examples, but the present invention is in no way limited to these examples.

### Evaluation Methods

### (1) Number-Average Fiber Diameter

Using a scanning electron microscope (Hitachi S-3400 N), an isolated fiber within the field of view was arbitrarily selected at a magnification of 350×, the fiber diameter was measured at ten points, and the number-average fiber diameter was recorded.

### (2) Nonwoven Fabric Properties

### (i) Thickness

Using an optical microscope (Keyence VHX-1000), thickness was measured (magnification: 50×; field of view: 7.0 mm×7.0) using a scale, and the numerical average of ten points was taken as the thickness of each sample.

### (ii) Basis Weight

100 mm×100 mm nonwoven fabric samples were die-cut and weighed. Ten samples were measured to calculate a numerical average, which was recorded as the basis weight.

### (i) Embossing Shape

Using an optical microscope (Keyence VHX-1000), the pitch of the non-embossed section, the width of the embossed line, and the embossed area proportion were measured (field of view: 10.0 mm×10.0 mm) using a scale. A nonwoven fabric sample was dried at 40° for 72 hours in a dry oven and observed using the height control menu from the depth up menu of the VHX menu of the same optical microscope. Upper and lower limits were set so as to include the most prominent part of the surface and the most recessed part of the surface of the nonwoven fabric. The depth-direction thickness of the nonwoven fabric sample was visualized using a photographing interval of 50 μm on the high pixel depth synthesis menu. Parts having 25% or less the thickness of the most prominent part of the surface were counted as embossed sections. The width of the embossed line and the pitch of the non-embossed section were measured using the scale included with the optical microscope at magnifications of 100×, 50×, and 25×.

Pitch:

A 150 mm (CD direction)×100 mm (MD direction) sized sheet was divided into four sections of equal area, and the pitch of five pairs of points a and b selected in the non-embossed sections of the different sections was measured according to the method described above. The length of a line segment drawn from a point a on an edge of the non-embossed section to another point b on another edge of the non-embossed section in a normal line direction to the first edge and passing only through the interior of the non-embossed section was measured. The line segment drawn from point a overlapped point b, and a line segment similarly drawn from point b along a normal line direction overlapped point a. The pitch of a total of twenty pairs of points a and b was obtained by measuring the four divided sections. The numerical average of the twenty values was calculated.

Embossed Line Width:

Ten pairs of points c and d were selected at uniform intervals along the lengthwise direction of the embossed line and width was measured according to the method described above. Specifically, the length of a line segment extending from a point c on an edge of the embossed line along a normal line direction to the edge through only the interior of the embossed line to another point d on another edge of the embossed line was measured. The line segment drawn from point c overlapped point d, and a line segment drawn from point d overlapped point c. The numerical average of the ten values was calculated.

Embossed Area Proportion:

Measurement was performed using the area measurement tool via the free line on the measurement menu.

### (ii) Break Elongation

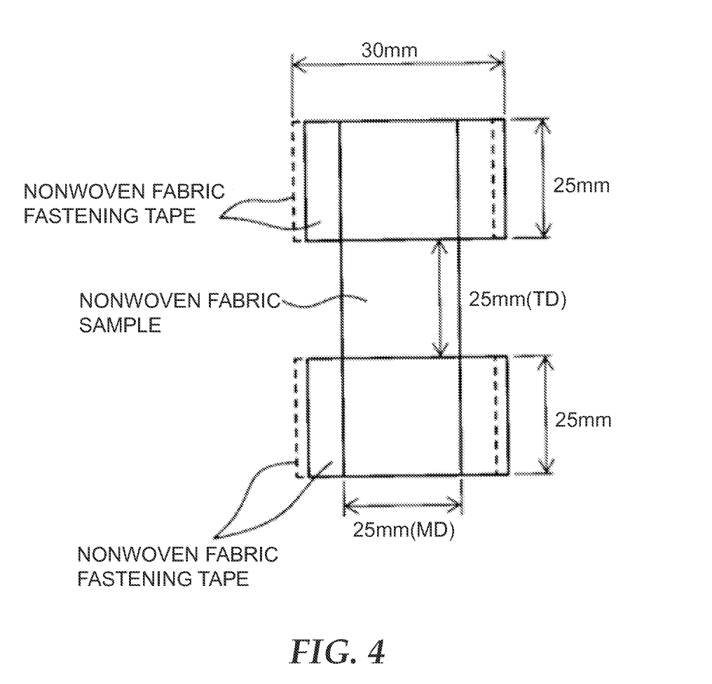

Break elongation was measured according to JIS L 1913 using a test piece having a width of 25 mm at a grip spacing of 25 mm and a tension rate of 300 mm/minute. FIG. 4 is an illustration of a method of measuring break elongation. A dried nonwoven fabric sample was prepared by drying in a dry oven at 40° C. for 72 hours. A test strip of width (MD) 25 mm and length (TD) 75 mm was cut out. The areas up to 25 mm from each end in the lengthwise direction was gripped using two pieces of 25 mm wide, 30 mm-long nonwoven fabric fastening tape (3M, Scotch™ Premium Grade Filament Tape; model: 898, 25 mm wide). The prepared test strip was fastened to a tensilon testing device (Orientec; model No. RTG-1225) at a grip spacing of 25 mm in a state of zero tension. The grips were set to a width of at least 25 mm and a height of at least 25 mm. The nonwoven fabric fastening tape of the test strip was affixed to the moving grip so as not to protrude from the lower end of the moving grip positioned above. A tensile test was performed by moving the moving grip up in the vertical direction at a rate of 300 mm/minute, and the break elongation (%) was recorded. The point at which the load was 0.2 N or less was detected as the breaking point. In the present example, each of different test strips cut from the same nonwoven fabric sample was measured as described above five times, and the numerical average obtained was taken as the break elongation (%).

### (3) Cleansing Sheet Evaluation

### (i) Wiped Amount

2 μL of foundation was dispensed dropwise onto a PP panel (100×50 mm), thoroughly spread out, and left standing at room temperature for 30 minutes, and a sheet impregnated with 0.45 L/sqm of a cleansing fluid was wrapped around the surface of a 100 g cylindrical roller. The sheet wrapped around the cylindrical roller was placed on one end of the PP panel and moved to the other end of the panel without rotating the roller (i.e., without altering the area of contact between the foundation and the sheet) to wipe off the foundation on the PP panel using the sheet. Similar wiping operations were performed a total of three times with different parts of the surface of the sheet in contact with the foundation on the PP panel. The total amount of foundation wiped off by the three wiping operations was quantized, and the amount of foundation wiped off by the sheet was rated according to the following standards.

Unacceptable: less than 45 wt. %
Acceptable: at least 45 wt. % and less than 60 wt. %
Good: at least 60 wt. % and less than 70 wt. %
Superior: 70 wt. % or greater

### (ii) Slipperiness (Sliminess)

The degree of slipperiness felt by 10 monitors when the cleansing sheet was actually used was rated according to the following standards.

1 point: extremely slippery
2 points: slippery
3 points: slightly slippery
4 points: no slipperiness

The ratings for the ten monitors were averaged and rated according to the following standards.

Less than 1.5 points: unacceptable
At least 1.5 points, less than 2.5 points: acceptable
At least 2.5 points, less than 3.5 points: good
3.5 point or greater: superior
(iii) Skin Feel (Scratching)

The skin feel felt by 10 monitors when the cleansing sheet was actually used was rated according to the following standards.

1 point: hard enough to be painful during use
2 points: somewhat hard; enough to be painful at times during use
3 points: soft; no pain during use
4 points: soft; extremely gentle on the skin

The ratings for the ten monitors were averaged and rated according to the following standards.

Less than 1.5 points: unacceptable
At least 1.5 points, less than 2.5 points: acceptable
At least 2.5 points, less than 3.5 points: good
3.5 point or greater: superior

### (iv) Nap

The degree of nap felt by 10 monitors when the cleansing sheet was actually used was rated according to the following standards.

1 point: extreme nap
2 points: some nap
3 points: slight nap
4 points: no nap

The ratings for the ten monitors were averaged and rated according to the following standards.

Less than 1.5 points: unacceptable
At least 1.5 points, less than 2.5 points: acceptable
At least 2.5 points, less than 3.5 points: good
3.5 point or greater: superior

### (v) Overall Rating

An overall rating for amount wiped off, degree of slipperiness, and skin feel was found according to the following standards.

Fail: There was a result of “unacceptable” for at least one of amount wiped off, slipperiness, and skin feel, or all three were no better than “acceptable”.
Pass: None of amount wiped off, slipperiness, and skin feel received a rating of “unacceptable”, and at least one received a rating of “good” or better.

### Examples 1 to 17

### Manufacturing and Rating Cleansing Sheets

The details of the fiber materials used for examples 1 to 16 shown in tables 1 and 2 were as follows.

Resin 1: Engage 8402 (polyolefin elastomer having a flexural modulus of 0.072 GPa, a melting point of 98.0° C., and a melt flow rate at 190° C. of 30 g/10 minutes; obtainable from Dow Chemical of Michigan, US)
Resin 2: Exxon Mobil LL 6201 (ethylene/butene copolymer having a flexural modulus of 0.28 GPa, a melting point of 123° C., and a melt flow rate at 190° C. of 50 g/10 minutes; obtainable from Exxon Mobil of Texas, US)
Resin 3: PLB00A (polypropylene having a flexural modulus greater than 1.0 GPa, a melting point of 130° C., and a melt flow rate at 190° C. of 70 g/10 minutes; obtainable from SunAllomer Ltd. of Tokyo, Japan)

Melt-blown nonwoven fabrics were produced from the abovementioned resins 1, 2, and 3 using a melt-blowing apparatus in general use in the industry. The resins were fed to an extruder having a temperature profile in which the temperature was gradually raised from an initial 220° C. to 335° C. The die temperature was kept at the temperature shown in table 1. While the resin was being extruded from the die (which was equipped with circular extrusion mouths) at a rate of 100 g/minute, hot air was blown in the die direction at the temperatures and pressures shown in table 1 to form resin fibers, which were trapped upon a screen to obtain a web.

Next, in the examples except for examples 1 and 6, the web was sent through an embossing line and embossed at the embossing temperatures and embossing pressures shown in table 1 (both settings being for the embossing rollers). The embossing rollers were a combination of an unpatterned back roller of hard rubber and an embossing roller having a raised pattern corresponding to the desired embossed shape. The rotational speed of the embossing roller and the hard rubber roller was 1 m/min. The obtained nonwoven fabrics were used as skin care sheets in examples 1 to 16.

The shapes and separation states of the non-embossed sections formed in the various examples are as shown in table 2. In table 2, “substantially separated” indicates that the non-embossed sections are substantially separated by the embossed section, and “completely separated” indicates that the non-embossed sections are completely discontinuously separated by a continuous embossed section. “Single” indicates that the non-embossed section provided on the nonwoven fabric comprises a single shape, “compressed section present” indicates that there are areas surrounded by the embossed section on the nonwoven fabric that, although not having been directly embossed, have a pitch that is less than 1.2 mm (pitch being defined identically as for the non-embossed sections), wherein the thickness of the nonwoven fabric is no more than 25% of the maximum thickness of the nonwoven fabric. Table 2 also indicates the drawing corresponding to the pattern shape of each example. Table 2 also indicates the width of the embossed line, the pitch of the non-embossed section, and the embossed area proportion.

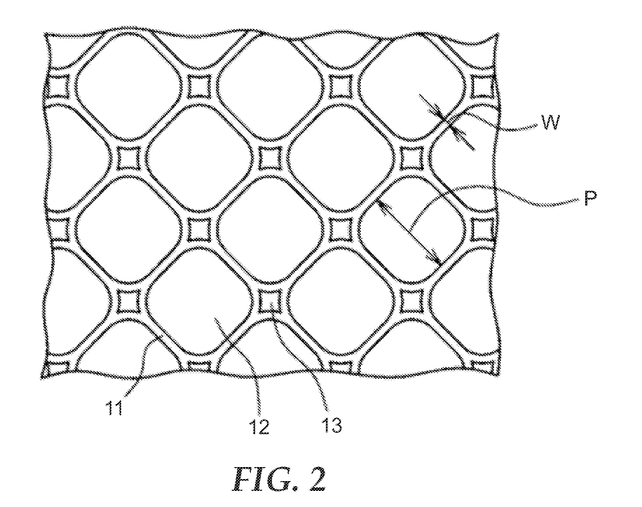

The pattern shape of comparative example 14 is shown in table 2. Referring to FIG. 2, comparative example 14 comprised a compressed section 13 which was considered an embossed section due to having a thickness equal to 25% or less of the maximum thickness of the nonwoven fabric, although it had not been directly contacted by the raised sections of the embossing roller.

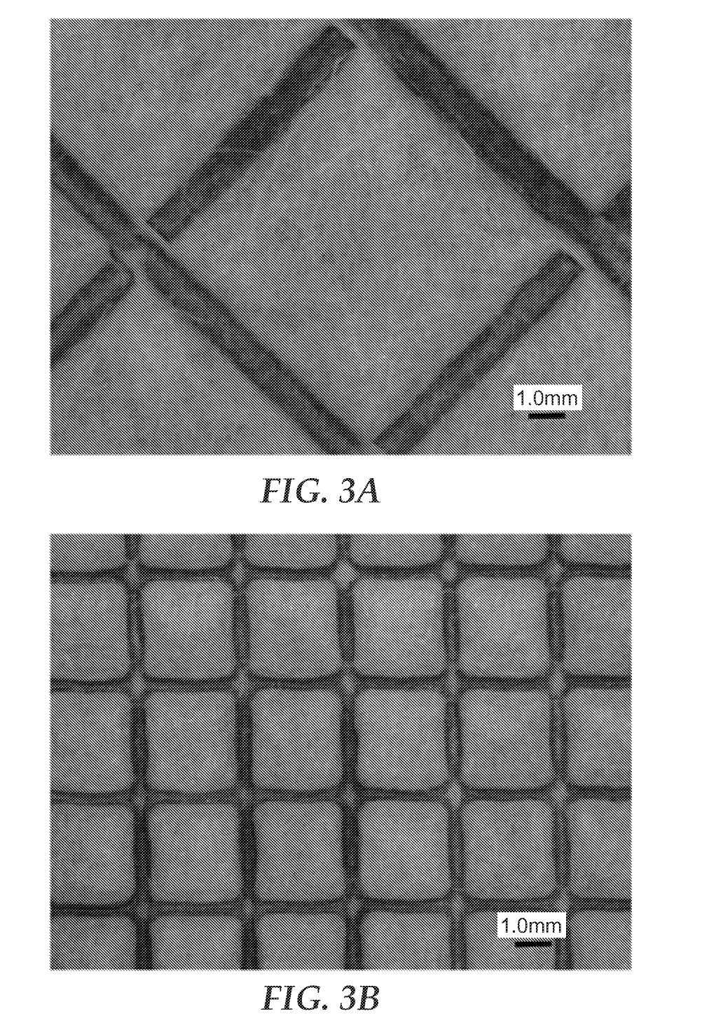

FIGS. 3A and 3B show microscopic images of the embossed shapes provided upon an example 7 (FIG. 3A), and an example 11 (FIG. 3B).

Meanwhile, a commercially available cotton nonwoven fabric was used as a skin care sheet in example 17.

Each of the skin care sheets was impregnated with commercially available cleansing liquids (water-based, using anionic surfactants) as liquid cosmetic components.

Evaluation results for the obtained cleansing sheets of examples 1 to 17 are shown in tables 3 to 5.

TABLE 1

| | | Fiber | Numerical | | | | | | | |
|---|---|---|---|---|---|---|---|---|---|---|
| | | material | average | | | | Sheet | | | Sheet |
| | | flexural | fiber | Die | Hot air | Sheet | basis | Embossing | | break |
| | Fiber | modulus | diameter | temperature | pressure | thickness | weight | temperature | | elongation |
| Example No. | material | (GPa) | (μm) | ° C. | (kgf/cm²) | (μm) | (g/mm²) | (° C.) | Embossing pressure | (%) |
| 1 (Comparative | Resin 1 | 0.072 | 5.8 | 320 | 0.1 | 673 | 62.1 | — | — | 402.2 |
| example) | | | | | | | | | | |
| 2 (Example) | Resin 1 | 0.072 | 5.8 | 320 | 0.1 | 923 | 63.0 | 85 | 3 MPa/150 × 100 mm | 402.2 |
| 3 (Comparative | Resin 1 | 0.072 | 16.2 | 320 | 0.1 | 896 | 57.0 | 85 | 3 MPa/150 × 100 mm | 687.7 |
| example) | | | | | | | | | | |
| 4 (Example) | Resin 2 | 0.28 | 8.4 | 370 | 0.2 | 863 | 65.0 | 85 | 3 MPa/150 × 100 mm | 255.43 |
| 5 (Comparative | Resin 3 | >1.0 | 5.7 | 330 | 0.3 | 543 | 57.8 | 85 | 3 MPa/150 × 100 mm | 87.6 |
| example) | | | | | | | | | | |
| 6 (Comparative | Resin 1 | 0.072 | 6.7 | 320 | 0.4 | 703 | 60.2 | | — | 369.4 |
| example) | | | | | | | | | | |
| 7 (Example) | Resin 1 | 0.072 | 6.7 | 320 | 0.4 | 793 | 60.2 | 85 | 3 MPa/150 × 100 mm | 369.4 |
| 8 (Example) | Resin 1 | 0.072 | 6.7 | 320 | 0.4 | 894 | 60.2 | 85 | 3 MPa/150 × 100 mm | 369.4 |
| 9 (Example) | Resin 1 | 0.072 | 6.7 | 320 | 0.4 | 906 | 60.2 | 85 | 3 MPa/150 × 100 mm | 369.4 |
| 10 (Example) | Resin 1 | 0.072 | 6.7 | 320 | 0.4 | 536 | 60.2 | 75 | 1.2 t/300 mm | 369.4 |
| 11 (Example) | Resin 1 | 0.072 | 6.7 | 320 | 0.4 | 492 | 60.2 | 70 | 1.2 t/300 mm | 369.4 |
| 12 (Example) | Resin 1 | 0.072 | 6.7 | 320 | 0.4 | 555 | 60.2 | 75 | 1.2 t/300 mm | 369.4 |
| 13 (Example) | Resin 1 | 0.072 | 6.7 | 320 | 0.4 | 602 | 60.2 | 80 | 1.2 t/300 mm | 369.4 |
| 14 (Comparative | Resin 1 | 0.072 | 6.7 | 320 | 0.4 | 692 | 60.2 | 85 | 0.8 t/300 mm | 369.4 |
| example) | | | | | | | | | | |
| 15 (Comparative | Resin 1 | 0.072 | 6.7 | 320 | 0.4 | 359 | 60.2 | 75 | 1.2 t/300 mm | 369.4 |
| example) | | | | | | | | | | |
| 16 (Comparative | Resin 1 | 0.072 | 6.7 | 320 | 0.4 | 361 | 60.2 | 75 | 0.8 t/300 mm | 369.4 |
| example) | | | | | | | | | | |
| 17 (Reference | Cotton | — | 26.6 | — | — | 367 | — | — | — | 159.3 |
| example) | | | | | | | | | | |
| 18 (Example) | Resin 1 | 0.072 | 6.7 | 320 | 0.4 | 677 | 60.2 | 80 | 1 MPa/150 × 100 mm | 369.4 |

TABLE 2

| | | | Non-embossed |
|---|---|---|---|
| | | Separation | section pitch |
| | | between | (mm)/embossed |
| | Non- | non-embossed | line width |
| | embossed | sections/shape | (mm)/ |
| | pattern | of non-embossed | embossed area |
| Example No. | shape | section | proportion (%) |
| 1 (Comparative example) | No | No embossing | No embossing |
| 2 (Example) | FIG. 1A | Substantially | 7.36/0.91/20.80 |
| | | separated/single | |
| 3 (Comparative example) | FIG. 1A | Substantially | 7.36/0.91/20.80 |
| | | separated/single | |
| 4 (Example) | FIG. 1A | Substantially | 7.36/0.91/20.80 |
| | | separated/single | |
| 5 (Comparative example) | FIG. 1A | Substantially | 7.36/0.91/20.80 |
| | | separated/single | |
| 6 (Comparative example) | No | No embossing | No embossing |
| 7 (Example) | FIG. 1A | Substantially | 7.53/0.74/17.10 |
| | | separated/single | |
| 8 (Example) | FIG. 1A | Substantially | 7.36/0.91/20.80 |
| | | separated/single | |
| 9 (Example) | FIG. 1A | Substantially | 7.08/1.30/28.62 |
| | | separated/single | |
| 10 (Example) | FIG. 1B | Completely | 2.52/0.38/18.27 |
| | | separated/single | |
| 11 (Example) | FIG. 1D | Completely | 2.32/0.40/27.25 |
| | | separated/single | |
| 12 (Example) | FIG. 1E | Completely | 4.01/0.16/13.28 |
| | | separated/ | |
| 13 (Example) | FIG. 1E | Completely | 3.82/0.27/23.66 |
| | | separated/ | |
| 14 (Comparative example) | FIG. 2 | Completely | 3.69/0.38/31.20 |
| | | separated/ | |
| 15 (Comparative example) | FIG. 1B | Completely | 1.09/0.21/29.70 |
| | | separated/single | |
| 16 (Comparative example) | FIG. 1B | Completely | 0.84/0.12/23.40 |
| | | separated/single | |
| 17 (Reference example) | No | No embossing | No embossing |
| 18 (Example) | FIG. 1F | Unseparated | 5.02/0.45/6.94 |

TABLE 3
Effects of fiber diameter on cleansing performance

| | Number- | | | | | | | |
|---|---|---|---|---|---|---|---|---|
| | average | | | | | | | |
| | fiber | | | | | | | |
| | diameter | Wiped amount | | | | | Overall | |
| Example No. | (μm) | Weight % | Rating | Slipperiness | Skin feel | Nap | rating | Remarks |
| 1 | 5.8 | 73.8 | Superior | Unacceptable | Superior | Unacceptable | Fail | No |
| (Comparative | | | | | | | | embossing |
| example) | | | | | | | | |
| 2 (Example) | 5.8 | 78.5 | Superior | Superior | Superior | Good | Pass | |
| 3 | 16.2 | 54.1 | Acceptable | Good | Unacceptable | Good | Fail | |
| (Comparative | | | | | | | | |
| example) | | | | | | | | |
| 17 (Reference | 26.7 | 43.7 | Unacceptable | Superior | Acceptable | Good | Fail | |
| example) | | | | | | | | |

TABLE 4
Effects of fiber material on cleansing performance

| | Fiber | Wiped amount | | | | | Overall |
|---|---|---|---|---|---|---|---|
| Example No. | material | Weight | Rating | Slipperiness | Skin feel | Nap | rating |
| 2 (Example) | Resin 1 | 78.5 | Superior | Good | Superior | Good | Pass |
| 4 (Example) | Resin 2 | 80.4 | Superior | Good | Acceptable | Good | Pass |
| 5 | Resin 3 | 56.2 | Acceptable | Superior | Unacceptable | Good | Fail |

TABLE 5
Effects of embossed shape upon cleansing performance

| | Non-embossed | | | | | | |
|---|---|---|---|---|---|---|---|
| | section pitch | | | | | | |
| | (mm)/ | | | | | | |
| | embossed line | | | | | | |
| | width (mm)/ | | | | | | |
| | embossed area | Wiped amount | | | | | Overall |
| Example No. | proportion (%) | Weight % | Rating | Slipperiness | Skin feel | Nap | rating |
| 4 (Example) | 7.36/0.91/20.80 | 80.4 | Superior | Good | Acceptable | Good | Pass |
| 6 | No embossing | 73.8 | Superior | Unacceptable | Good | Unacceptable | Fail |
| (Comparative | | | | | | | |
| example) | | | | | | | |
| 7 (Example) | 7.53/0.74/17.10 | 75.7 | Superior | Good | Superior | Good | Pass |
| 8 (Example) | 7.36/0.91/20.80 | 78.5 | Superior | Good | Superior | Good | Pass |
| 9 (Example) | 7.08/1.30/28.62 | 70.1 | Superior | Acceptable | Acceptable | Good | Pass |
| 10 (Example) | 2.52/0.38/18.27 | 75.7 | Superior | Good | Good | Good | Pass |
| 11 (Example) | 2.32/0.40/27.25 | 73.2 | Superior | Superior | Superior | Good | Pass |
| 12 (Example) | 4.01/0.16/13.28 | 77.5 | Superior | Good | Superior | Acceptable | Pass |
| 13 (Example) | 3.82/0.27/23.66 | 73.5 | Superior | Acceptable | Good | Good | Pass |
| 14 | 3.69/0.38/31.20 | 49.8 | Acceptable | Acceptable | Acceptable | Good | Fail |
| (Comparative | | | | | | | |
| example) | | | | | | | |
| 15 | 1.09/0.21/29.70 | 39.4 | Unacceptable | Superior | Unacceptable | Superior | Fail |
| (Comparative | | | | | | | |
| example) | | | | | | | |
| 16 | 0.84/0.12/23.40 | 41.5 | Unacceptable | Superior | Unacceptable | Superior | Fail |
| (Comparative | | | | | | | |
| example) | | | | | | | |
| 17 | Cotton (no | 43.7 | Unacceptable | Superior | Acceptable | Good | Fail |
| (Reference | embossing) | | | | | | |
| example) | | | | | | | |
| 18 (Example) | 5.02/0.45/6.94 | 78.8 | Superior | Acceptable | Superior | Acceptable | Pass |

### REFERENCE NUMERALS

11 Embossed section
12, 12a, 12b Non-embossed section
13 Compressed section

1. A skin care sheet comprising a nonwoven fabric;
the nonwoven fabric having a break elongation of at least 250%;
the nonwoven fabric comprising an embossed section and a non-embossed section defined by the embossed section, the non-embossed section having a pitch of 1.2 to 10.0 mm;
a proportion of a total area of the embossed section occupying from 5 to 30% of a total area of the nonwoven fabric on at least one surface of the nonwoven fabric;
fibers present in the nonwoven fabric having a number-average fiber diameter of 0.1 to 15.0 μm; and
the fibers containing a resin having a flexural modulus of 0.5 GPa or less.

2. The skin care sheet according to claim 1, wherein the nonwoven fabric is single-layered.

3. The skin care sheet according to claim 1, wherein the embossed section is an embossed line.

4. The skin care sheet according to claim 3, wherein the embossed line has a width of 0.1 to 1.3 mm.

5. The skin care sheet according to claim 1, wherein the embossed section separates a plurality of the non-embossed sections.

6. A skin care article comprising the skin care sheet according to claim 1, and a liquid cosmetic component.

7. The skin care article according to claim 6, wherein the skin care article is a cleansing sheet.
